(19) United States
(12) Patent Application Publication
(10) Pub. No.: US 2026/0137316 A1
(43) Pub. Date: May 21, 2026

(54) Blood Collection Device Including Pain Reduction Features

(71) Applicant: Becton, Dickinson and Company, Franklin Lakes, NJ (US)

(72) Inventors: Kishore K. Bokka Srinivasa Rao, Lyndhurst, NJ (US); Leslie Porschen, Clifton, NJ (US); Alyse Danielle Krausz, Durham, NC (US); Anthony V. Torris, Mercer Island, WA (US)

(21) Appl. No.: 19/121,443

(22) PCT Filed: Oct. 24, 2023
(86) PCT No.: PCT/US2023/077594

§ 371 Date: Apr. 16, 2025

Related U.S. Application Data
(60) Provisional application No. 63/419,123, filed on Oct. 25, 2022.

(51) Int. Cl. A61B 5/15

(52) U.S. Cl. CPC A61B 5/150106; A61B 5/150022; A61B 5/150068; A61B 5/150748

(57) ABSTRACT

A device for obtaining a blood sample, the device including a holder for receiving a sample source, the holder having an actuation portion and a port, a container engagement portion connected to the holder; and a pain reduction member provided on an inner surface of the holder.

### CROSS-REFERENCE TO RELATED APPLICATION

The present application claims priority to U.S. Provisional Patent Application No. 63/419,123 entitled “Blood Collection Device Including Pain Reduction Features” filed Oct. 25, 2022, the entire disclosure of which is hereby incorporated by reference in its entirety.

### BACKGROUND OF THE INVENTION

### Field of the Invention

The present disclosure relates generally to devices for obtaining a biological sample. More particularly, the present disclosure relates to finger-based capillary blood collection devices, which can be provided with features to provide comfort to patient's fingers during use of the blood collection device.

### Description of Related Art

Devices for obtaining and collecting biological samples, such as blood samples, are commonly used in the medical industry. One type of blood collection that is commonly done in the medical field is capillary blood collection, which is often done to collect blood samples for testing. Certain diseases, such as diabetes, require that a patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed, which are used for puncturing the skin of a patient to obtain a capillary blood sample from the patient.

Many different types of lancet devices are commercially available to hospitals, clinics, doctors'offices, and the like, as well as to individual consumers. Such devices typically include a sharp-pointed member, such as a needle, or a sharp-edged member, such as a blade, that is used to make a quick puncture wound or incision in the patient's skin in order to provide a small outflow of blood. In order to simplify capillary blood collection, lancet devices have evolved into automatic devices that puncture or cut the skin of the patient upon actuation of a triggering mechanism. In some devices, the needle or blade is kept in a standby position until it is triggered by the user. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position, wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Use of lancet devices for capillary blood collection can be complex requiring a high skill level for the healthcare worker performing the blood collection procedure. The multi-step nature of the capillary blood collection process can introduce several variables that may cause sample quality issues, such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

The general population has various finger sizes. A patient's finger size may be dimensioned between two different sizes for a blood collection device or the patient's fingers may be too small or too large for the specific blood collection device. While capillary blood collection devices and assemblies have been developed to simplify the capillary blood collection processes, such as finger-based capillary blood collection devices configured to lance and squeeze a finger, collect, stabilize, and dispense a blood sample in a controlled manner, problems related to blood sample quality can still occur. Attempting to perform a blood draw procedure using a device that is not correctly sized for a patient's finger can exacerbate such blood sample quality problems and can create discomfort for the patient's finger. For example, an incorrectly sized blood collection device may apply pressure to the patient's finger in an imprecise or inconsistent manner, resulting in poor blood flow from the lanced finger and soreness or pain on the patient's finger. The blood collection devices and methods of the present disclosure are configured to improve patient comfort for blood collection devices to improve blood sample quality.

### SUMMARY OF THE INVENTION

In one non-limiting embodiment or aspect of the present disclosure, a device for obtaining a blood sample may include a holder for receiving a sample source, the holder having an actuation portion and a port; a container engagement portion connected to the holder; and a pain reduction member provided on an inner surface of the holder.

In one non-limiting embodiment or aspect of the present disclosure, the pain reduction member may include at least one elastomeric material positioned on the inside surface of the holder. The pain reduction member may be positioned on an inner surface of the actuation portion of the holder. The at least one elastomeric material may be positioned to contact a dorsal portion of the sample source held in the holder. The pain reduction member may include a ring configured to cover an outer circumferential surface of the sample source. The ring may be made of an elastomeric material. The ring may include a plurality of ribs or bumps on an exterior surface thereof to grip the holder. The sample source may be a finger. The finger may be received within the holder, the port is in communication with a portion of the finger. The actuation portion may include a pair of opposing wings that are configured to be pumped to draw the blood sample from the sample source.

In one non-limiting embodiment or aspect of the present disclosure, a device for obtaining a blood sample may include a holder for receiving a sample source, the holder having an actuation portion and a port; a container engagement portion connected to the holder; and a tightening arrangement provided on the holder to adjust a diameter of the holder that receives the sample source.

In one non-limiting embodiment or aspect of the present disclosure, the tightening arrangement may include a zip tie to decrease the diameter to the holder to tighten the holder to the sample source. The zip tie may include a zip tie strap on a first portion of the holder and a zip tie opening provided on a second portion of the holder. The tightening arrangement may be provided on a bottom surface of the holder. The sample source may be a finger. With the finger received within the holder, the port may be in communication with a portion of the finger. The actuation portion may include a pair of opposing wings that are configured to be pumped to draw the blood sample for the sample source. The actuation portion may include a pumping member for applying pressure to the sample source.

In one non-limiting embodiment or aspect of the present disclosure, a kit used for obtaining a blood sample, the kit may include a blood collection device comprising a holder for receiving a sample source, the holder having an actuation portion and a port; and a container engagement portion connected to the holder; and a pain reduction member provided separate from the blood collection device, the pain reduction member configured to minimize or eliminate pain experienced by the sample source held in the holder. The pain reduction member may include an elastomeric ring configured to surround an outer circumferential surface of the sample source.

The present invention is also disclosed in the following clauses:
Clause 1: A device for obtaining a blood sample, the device comprising a holder for receiving a sample source, the holder having an actuation portion and a port; a container engagement portion connected to the holder; and a pain reduction member provided on an inner surface of the holder.
Clause 2: The device of clause 1, wherein the pain reduction member comprises at least one elastomeric material positioned on the inner surface of the holder.
Clause 3: The device of Clause 1 or Clause 2, wherein the pain reduction member is positioned on an inner surface of the actuation portion of the holder.
Clause 4: The device of Clause 2, wherein the at least one elastomeric material is positioned to contact a dorsal portion of the sample source held in the holder.
Clause 5: The device of any of Clauses 1-4, wherein the pain reduction member comprises a ring configured to cover an outer circumferential surface of the sample source.
Clause 6: The device of Clause 5, wherein the ring is made of an elastomeric material.
Clause 7: The device of Clause 5 or Clause 6, wherein the ring comprises a plurality of ribs or bumps on an exterior surface thereof to grip the holder.
Clause 8: The device of any of Clauses 1-7, wherein the sample source is a finger.
Clause 9: The device of Clause 8, wherein, with the finger received within the holder, the port is in communication with a portion of the finger.
Clause 10: The device of any of Clauses 1-9, wherein the actuation portion comprises a pair of opposing wings that are configured to be pumped to draw the blood sample from the sample source.
Clause 11: A device for obtaining a blood sample, the device comprising a holder for receiving a sample source, the holder having an actuation portion and a port; a container engagement portion connected to the holder; and a tightening arrangement provided on the holder to adjust a diameter of the holder that receives the sample source.
Clause 12: The device of Clause 11, wherein the tightening arrangement includes a zip tie to decrease the diameter of the holder to tighten the holder to the sample source.
Clause 13: The device of Clause 12, wherein the zip tie includes a zip tie strap on a first portion of the holder and a zip tie opening provided on a second portion of the holder.
Clause 14: The device of any of Clauses 11-13, wherein the tightening arrangement is provided on a bottom surface of the holder.
Clause 15: The device of any of Clauses 11-14, wherein the sample source is a finger.
Clause 16: The device of Clause 15, wherein, with the finger received within the holder, the port is in communication with a portion of the finger.
Clause 17: The device of any of Clauses 11-16, wherein the actuation portion comprises a pair of opposing wings that are configured to be pumped to draw the blood sample for the sample source.
Clause 18: The device of any of Clauses 11-17, wherein the actuation portion includes a pumping member for applying pressure to the sample source.
Clause 19: A kit used for obtaining a blood sample, the kit comprising a blood collection device comprising: a holder for receiving a sample source, the holder having an actuation portion and a port; and a container engagement portion connected to the holder; and a pain reduction member provided separate from the blood collection device, the pain reduction member configured to minimize or eliminate pain experienced by the sample source held in the holder.
Clause 20: The kit of Clause 19, wherein the pain reduction member comprises an elastomeric ring configured to surround an outer circumferential surface of the sample source.

### BRIEF DESCRIPTION OF THE DRAWINGS

FIG. 1A is a perspective view of a capillary blood collection device for obtaining a blood sample from a patient's finger and a collection container, according to an aspect of the present disclosure.

FIG. 1B is a cross-sectional view of a capillary blood collection device and a lancet, according to an aspect of the present disclosure.

FIG. 1C is a perspective view of a holder of a capillary blood collection device, according to an aspect of the present disclosure.

FIG. 1D is a schematic drawing showing a top view of the holder of FIG. 1C connected to a patient's finger for performing a blood collection procedure.

FIG. 1E is another schematic drawing showing a front view of the holder of FIG. 1C connected to the patient's finger.

FIG. 2 is a front view of a capillary blood collection device including pain reduction features, according to an aspect of the present disclosure.

FIG. 3 is a front view of a capillary blood collection device including a finger cuff and an assembly of different finger cuff sizes, according to an aspect of the present disclosure.

FIG. 4 is a front view of a capillary blood collection device including a tightening feature, according to an aspect of the present disclosure.

FIG. 5 is a view of a capillary blood collection device including a tightening feature, according to an aspect of the present disclosure.

FIG. 6 is a view of a capillary blood collection device including a tightening feature, according to an aspect of the present disclosure.

FIG. 7 is a view of a capillary blood collection device including a tightening feature, according to an aspect of the present disclosure.

FIG. 8 is a view of a capillary blood collection device including a tightening feature, according to an aspect of the present disclosure.

FIG. 9A is a view of a capillary blood collection device including a tightening feature, according to an aspect of the present disclosure.

FIG. 9B is a view of a capillary blood collection device including a tightening feature, according to an aspect of the present disclosure.

FIG. 10 is a view of a capillary blood collection device including a tightening feature, according to an aspect of the present disclosure.

FIG. 11 is a view of a capillary blood collection device including a tightening feature, according to an aspect of the present disclosure.

FIG. 12 is a view of a capillary blood collection device including a tightening feature, according to an aspect of the present disclosure.

FIG. 13 is a view of a capillary blood collection device including a tightening feature, according to an aspect of the present disclosure.

FIG. 14 is a view of a capillary blood collection device including a tightening feature, according to an aspect of the present disclosure.

### DESCRIPTION OF THE DISCLOSURE

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

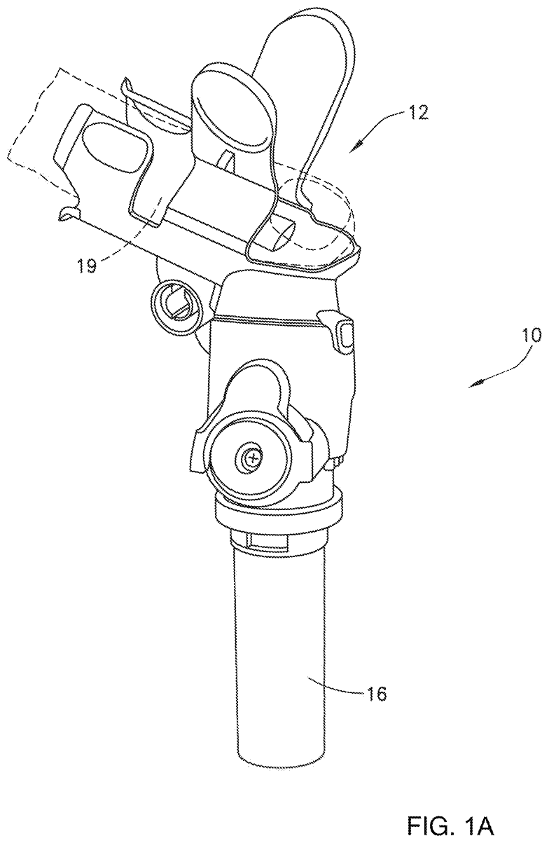

The present disclosure is directed to blood collection devices 10 (shown in FIGS. 1A-E) that include pain reduction features to assist in comforting a patient's finger during use of the blood collection devices 10. As will be appreciated by those skilled in the art, for many products that come in different sizes, such as clothing, there are typically no safety concerns with having multiple people try on the product for fit. Further, if there is a concern about proper sizing, a user or wearer can dispose of the product rather than continuing to use an incorrectly sized product. However, for medical devices, such as blood collection devices 10, there can be safety concerns with using a device 10 of an improper size for a patient's finger. Further, disposing of medical devices, such as a blood collection device 10, because the devices are not a suitable size for a particular patient is inefficient and increases costs of such devices and medical procedures using the devices 10. The devices, systems, and methods disclosed herein are intended to improve the comfort of use for blood collection devices 10, as well as to help ensure that a blood collection device 10 being used for each patient is comfortable regardless of the size of the patient's finger.

According to one aspect, the present disclosure relates to methods for designing and/or manufacturing capillary blood collection devices 10 including finger cuffs or holders 12 that include features that reduce or eliminate pain created or caused by using the blood collection device 10 on a patient's finger. For example, if the holder 12 or finger cuff is too loose, the holder 12 or cuff may move or fall off during a blood collection procedure. If the holder 12 or finger cuff is too tight, the device 10 may restrict blood flow or cause pain for the patient. During pumping of the blood collection device 10, portions of the blood collection device 10 contact and press on the patient's finger. Each of these circumstances can cause pain or discomfort on a patient's finger during use of the blood collection device 10.

### Blood Collection Device

Examples of blood collection devices 10 having dimensions that can be optimized to correctly fit patients within defined size ranges are shown in FIGS. 1A-1E. The blood collection device 10 can be, for example, a self-contained and fully integrated finger-based capillary blood collection device with the ability to lance, collect, and stabilize a high volume capillary blood sample, e.g., up to or above 500 microliters. The blood collection device 10 can also be a device formed from separable components (i.e., a finger cuff or holder 12, lance, and sample container) that can be connected and/or used together to obtain a blood sample. Some additional exemplary capillary blood collection devices and assemblies that can be modified for use with the systems and methods of the present disclosure are described in United States Patent Appl. Pub. No. 2019/0216380, entitled “Device for Obtaining a Blood Sample”; United States Patent Appl. Pub. No. 2019/0223772, entitled “Device for the Attached Flow of Blood”; and PCT Publication No. WO 2020/167746, entitled “Capillary collector with rotatable connection,” each of which is incorporated herein by reference in its entirety.

With reference to FIGS. 1A and 1B, the example blood collection device 10 includes the integrated holder 12 (also referred to herein as the finger cuff), a lancet housing or lancet 14 (shown in FIG. 1B) for puncturing a finger 19 (shown in FIGS. 1D and 1E) of the patient, and a collection container 16 (shown in FIG. 1A). In other examples, the blood collection device 10 can be provided as a semi-integrated device 10 including, for example, an integrated lancet housing 14 and collection container 16 that can be connected with a separate holder 12. In other examples, a semi-integrated device may have an in-line flow and an integrated lancet housing and collection container, which can be connected with a separate holder.

The holder 12 is configured to receive a sample source, e.g., the finger 19 of a patient, for supplying a biological sample, such as a blood sample. As shown in FIGS. 1C-1E, the holder 12 generally includes a finger receiving portion 20 having a first opening 22, an actuation portion 24, a port 26 having a second opening 28, and a finger end guard 30. The first opening 22 can have a width W1 (shown in FIGS. 1D and 1E), which corresponds to a maximum finger width for a finger 19 that can be used with the device 10. The first opening 22 can also have a height H1 (shown in FIG. 1E), which corresponds to a maximum finger height for a finger 19 that can be used with the device 10. The finger receiving portion 20 can also have a length L1 (shown in FIG. 1D), which corresponds to a maximum finger length (i.e., finger length from the fingertip to the second knuckle) of a finger 19 that can be used with the device 10. If a patient's finger 19 is greater than the width W1, height H1, and/or length L1, then a different size of blood collection device 10 and/or holder 12 should be used for the patient.

The finger end guard 30 is configured to provide a stop portion for properly aligning and securing the finger 19 within the holder 12. The finger end guard 30 further assists in ensuring the patient's finger 19 is placed at a proper position within the finger receiving portion 20 so that applied pressure to the patient's finger 19 will result in adequate blood flow. The finger end guard 30 can have a curved fingertip rest that ensures the patient's finger 19 stops at an end of the finger receiving portion 20, while permitting the patient's finger nail to clear the end of the finger receiving portion 20. The finger receiving portion 20 permits use of the holder 12 with artificial and natural fingernail styles present in the patient population.

The first opening 22, having the width W1 and the height H1, is configured for receiving the sample source, e.g., the finger 19. The sample source may also include other parts of the body capable of fitting within the first opening 22, such as toes or other extremities. The port 26 is in communication with the finger receiving portion 20. For example, with a finger 19 received within the holder 12, the port 26 is in communication with a portion of the finger 19. As described in further detail herein, the holder 12 can be sized for use with a particular subset of patients. For example, a small holder 12 may be an appropriate size of a bottom quartile of patients. Medium and large-sized holders 12 can be sized for use with patients in the lower middle quartile of patients (25% to 50%) and the upper middle quartile of patients (50% to 75%), respectively.

The second opening 28 of the port 26 is configured for receiving the lancet housing or lancet 14 (shown in FIG. 1B) and a collection container 16 (shown in FIG. 1A). In some examples, the port 26 further includes a locking portion 32 for securely receiving the lancet housing or lancet 14 and the collection container 16 within the port 26.

The actuation portion 24 of the device 10 is transitionable between a first position, in which the holder 12 defines a first diameter, and a second position, in which the holder 12 defines a second diameter, with the second diameter being less than the first diameter. Further, in the first position, the holder 12 defines a first elliptical shape or cross-section. In the second position, the holder 12 defines a second elliptical shape or cross-section, with the first elliptical shape or cross-section being different than the second elliptical shape or cross-section. In this manner, with the holder 12 in the second position with a reduced diameter, a portion of the holder 12 contacts the sample source (i.e., the finger 19) and the actuation portion 24 of the holder 12 is able to pump and/or extract blood as described in more detail below.

In some examples, the actuation portion 24 includes a contact member 34. With the actuation portion 24 in the first position, the contact member 34 is in a disengaged position, i.e., the contact member 34 is provided in a first position with respect to the sample source, such that the contact member 34 may be in slight contact therewith. With the actuation portion 24 in the second position, the contact member 34 is in an engaged position, i.e., the contact member 34 is provided in a second position with respect to the finger 19, such that the contact member 34 is in an applied pressure contact with the finger 19, and the actuation portion 24 of the holder 12 is able to pump and/or extract blood. For example, with the contact member 34 in the engaged position, the contact member 34 exerts a pressure on the sample source.

In some examples, the actuation portion 24 includes a pumping member 36 for applying pressure to the finger 19, such as a pair of opposed tabs or wings 38. Each wing 38 can include a contact member 34. The holder 12 can also include a living hinge portion 42. The living hinge portion 42 allows the user to squeeze the wings 38 between a first position (passive state) and a second position (active state). It is believed that use of the tabs or wings 38 to draw blood out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12, while flexing to accommodate the largest patient finger within a holder 12 without blood occlusion. In some examples, the wings 38 may be positioned on the finger receiving portion 20 at a position located proximal of a patient's fingernail and distal of a patient's first knuckle to avoid hard tissues on the patient's finger 19.

The holder 12 can be configured to allow a user to repeatedly squeeze and release the wings 38 to pump and/or extract blood from a finger 19 until a desired amount of blood is filled in a collection container 16. The wings 38 are configured to flex to maintain gentle contact with a range of patient finger sizes that may be used with the holder 12 and to retain the holder 12 on the patient's finger 19. The wings 38 may also provide active pressure features for the holder 12.

In some examples, the holder 12 can include a stability extension portion 40. This provides additional support for the holder 12 to be securely placed onto the finger 19. In one example, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

The blood collection device 10 for obtaining the blood sample also includes the lancet housing or lancet 14 (shown in FIG. 1B) that is removably connectable to the port 26 of the holder 12. Referring to FIG. 1B, the lancet housing or lancet 14 can include an inlet or opening 50, an interior 52, a puncturing element 54, an engagement portion 56, a retractable mechanism 58, and a drive spring 60. The puncturing element 54 can be moveable between a pre-actuated position, wherein the puncturing element 54 is retained within the interior 52 of the lancet housing 14, and a puncturing position, wherein at least a portion of the puncturing element 54 extends through the inlet 50 of the lancet housing or lancet 14 to lance a portion of a finger 19. In one example, the lancet 14 of the present disclosure is a contact activated lancet and may be constructed in accordance with the features disclosed in U.S. Pat. No. 9,380,975, entitled “Contact Activated Lancet Device”, which is incorporated herein by reference in its entirety.

In some examples, the holder 12 and the lancet housing or lancet 14 are separate components that can be removably connectable to the port 26 of the holder 12. In such examples, the lancet housing or lancet 14 includes the engagement portion 56. The lancet housing or lancet 14 can be pushed into the port 26 of the holder 12, such that the engagement portion 56 of the lancet housing or lancet 14 is locked within the locking portion 32 of the holder 12. In this manner, the lancet housing 14 is securely connected and locked to the holder 12, such that the puncturing element 54 of the lancet housing 14 can be activated to lance or puncture a sample source, e.g., the finger 19. In some examples, the port 26 of the holder 12 includes a plurality of ribs for securing and locking the lancet 14 or the collection container 16 in the port 26.

To activate the lancet 14, the lancet 14 is pushed against the finger 19 to activate the retractable mechanism 58 and drive spring 60 of the lancet 14 to lance the finger 19. After puncturing, the puncturing element 54 is immediately retracted and safely secured within the interior 52 of the lancet housing 14. Once the finger 19 is punctured, the blood sample is squeezed from the finger 19 into a collection container 16. The collection container 16 may also contain a sample stabilizer, e.g., an anticoagulant, to stabilize a blood sample and/or a component of a blood sample disposed therein. The collection container 16 may also include at least one fill line(s) corresponding to a predetermined volume of sample. The collection container 16 may also indicate/meter a collected volume of blood.

In order to use the capillary blood collection device 10 shown in FIGS. 1A-1E, a desired finger 19 is first cleaned and a holder 12 having an appropriate size for the desired finger 19 is selected and placed onto the finger 19 securely. Next, the lancet housing or lancet 14 is connected to the port 26 of the holder 12. As discussed previously, the lancet housing or lancet 14 is pushed into the port 26 of the holder 12, such that the engagement portion 56 of the lancet housing or lancet 14 is locked within the locking portion 32 of the holder 12. In this manner, the lancet housing or lancet 14 is securely connected and locked to the holder 12, such that the puncturing element 54 of the lancet housing 14 can be activated to lance or puncture the finger 19. With the lancet 14 connected to the port 26 of the holder 12, the lancet 14 is in communication with the finger 19.

When it is desired to activate the lancet 14 to lance the skin of the finger 19, the lancet 14 is pushed against a finger 19 to activate a retractable mechanism 58 of the lancet 14 to lance the finger 19. After the finger 19 is lanced to create blood flow from the finger 19, the lancet 14 is removed from the holder 12 and the collection container 16 is pushed into the port 26 of the holder 12. With the container 16 properly secured to the holder 12 for collection of a blood sample, the user repeatedly squeezes and releases the wings 38 of the holder 12 to pump and/or extract blood from the finger 19 until a desired amount of blood is collected in the collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Once a desired amount of blood is collected within the container 16, a blood collector portion including the collection container 16 can be detached from the collection device 10 in order to send a collected sample to a diagnostic instrument and/or testing device. The blood collector portion can be sealed via the cap or septum once removed from the collection device 10 to protectively seal the blood sample within the collection container 16.

### Pain Reduction or Elimination Features

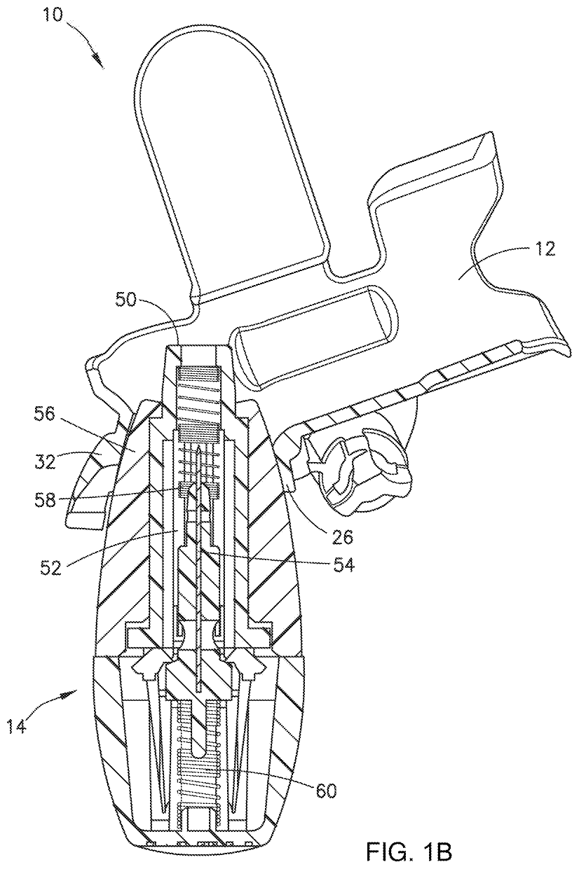

Due to the contact of the wings 38 against the patient's finger, a patient may experience pain or discomfort on a dorsal surface of his/her finger due to the constant contact from the wings 38. It is also contemplated that the patient may experience pain or discomfort on a bottom surface of his/her finger due to the pressure of the wings 38 pushing the patient's finger into the holder 12. With reference to FIG. 2, according to one aspect or example of the present disclosure, the blood collection device 10 may include at least one pain reduction member 100A, 100B to reduce or eliminate the pain or discomfort experienced by the patient when using the blood collection device 10. In one example, the pain reduction member 100A, 100B may be positioned on an inner surface of the blood collection device 10 with at least a portion positioned on the wings 38 and at least a portion positioned on the holder 12 to contact the patient's finger 19 when held in the blood collection device 10. In another example, the pain reduction member 100A, 100B may be positioned on the entire inner surface of the wings 38 and the holder 12 to contact all portions of the patient's finger 19 that contact the blood collection device 10. In one example, the blood collection device 10 may include a first pain reduction member 100A positioned on a first wing 38 and a second pain reduction member 100B positioned on a second wing 38 such that the pain reduction members 100A, 100B are positioned to contact opposing sides of the patient's finger 19.

The pain reduction members 100A, 100B may be made of an elastomeric material that provides a cushion for the patient's finger 19 to rest against when the wings 38 are being pumped. In one example, the pain reduction members 100A, 100B may be elastomeric pads, patches, or strips that are removably attached to the inner surface of the wings 38 and the holder 12. The pain reduction members 100A, 100B may also be permanently secured to the wings 38 and the holder 12 using an adhesive, by welding, or any other similar connection method. In another example, the pain reduction members 100A, 100B may be integrally formed with the wings 38 and holder 12. The pain reduction members 100A, 100B may be configured to absorb concentrated stress from the wings 38 and minimize or eliminate the pain experienced by the patient. The pain reduction members 100A, 100B may be configured to reduce stress and pain on the dorsal or top portion of the patient's finger 19.

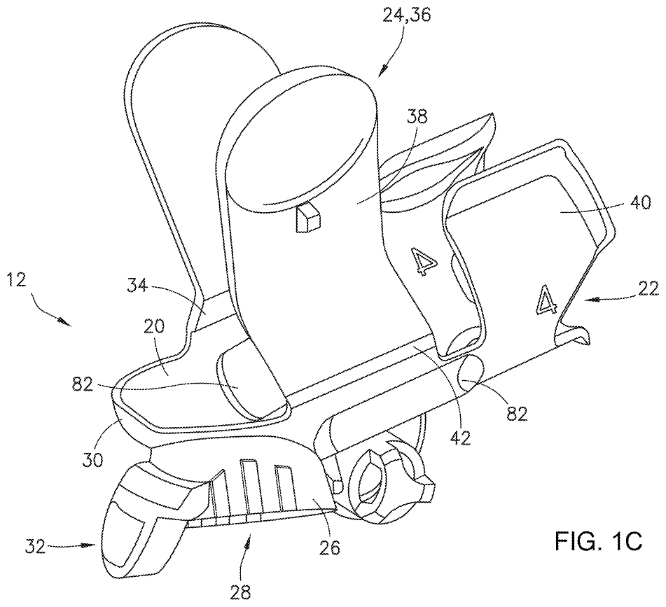

With reference to FIG. 3, according to a non-limiting aspect or example of the present disclosure, the blood collection device 10 may include a pain reduction member 102 that may be configured to reduce or eliminate pain on the patient's finger 19 due to the patient's finger 19 being too small for the size of the blood collection device 10 being used by the patient. The pain reduction member 102 may be used to in the event the patient's finger 19 is not the suggested size for the particular blood collection device 10 being used by the patient. In one example, the pain reduction member 102 may be an elastomeric sleeve or ring that is fitted on the patient's finger 19 to act as a cushion between the patient's finger 19 and the holder 12. The pain reduction member 102 may be used for patient's finger 19 that are either between sizes for the blood collection device 10 or patient's fingers 19 that are too small to fit into the small cuff size for the blood collection device 10. The pain reduction member 102 may be ring shaped to cover the entire circumference of the patient's finger 19. In one example, the pain reduction member 102 may be configured to reduce or eliminate pain on a dorsal or top portion of the patient's finger 19. The pain reduction member 102 may also include ribs or bumps on an exterior surface to assist in gripping the blood collection device 10 when the patient's finger 19 is positioned in the holder 12. In another example, a kit may be provided as the blood collection device 10 and, separately, the pain reduction member 102.

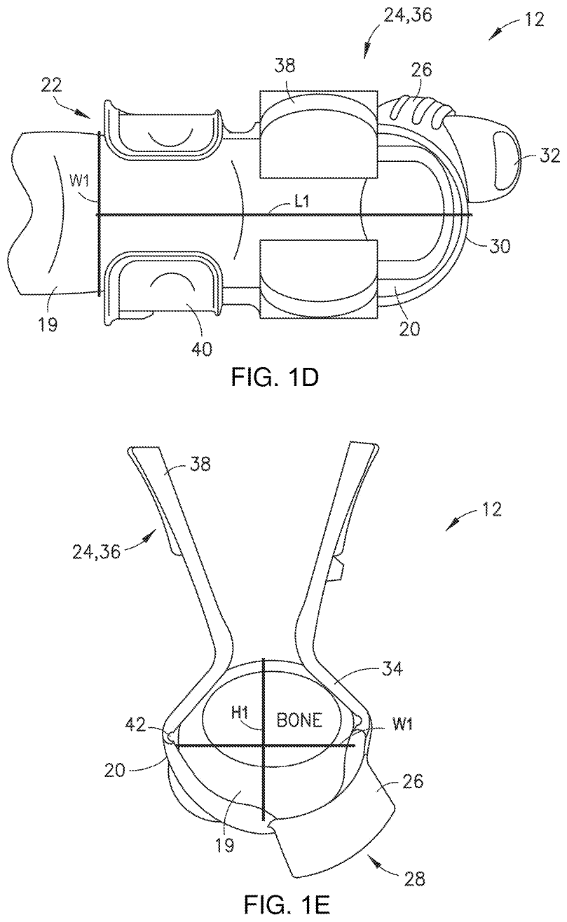

With reference FIG. 4, according to a non-limiting aspect or example of the present disclosure, the blood collection device 10 may include a tightening arrangement 104 that may assist in reducing or eliminating pain on the patient's finger 19 during use of the blood collection device 10. The tightening arrangement 104 may be configured to tighten the holder 12 once the patient's finger 19 is positioned in the holder 12. By tightening the holder 12 to the patient's finger 19, a pressure from the wings 38 and portions of the holder 12 when the blood collection device 10 is being used is reduced. Since the wings 38 do not need to be pressed inwardly as far as when the holder 12 is not tightened against the patient's finger 19, less force is required to press against the patient's finger 19, thereby reducing or eliminating the pain on the patient's finger 19. The tightening arrangement 104 may be used when the patient's finger 19 is too small for the blood collection device 10 that is being used by the patient. In one example, by tightening the holder 12, the diameter of the holder 12 is reduced to accommodate a smaller patient finger 19. The tightening arrangement 104 may include a zip tie mechanism including a zip tie strip 106 and a zip tie opening 108 to receive the zip tie strip 106. The zip tie strip 106 can be pulled through the zip tie opening 108 to tighten the holder 12 against the patient's finger 19. In another example, instead of the zip tie mechanism, the holder 12 can include hook and loop strips to tighten the holder 12 against the patient's finger 19. Using the tightening arrangement 104 with the blood collection device 10, the blood collection device 10 can be adjusted to accommodate any size patient finger 19, thus eliminating the need to size the blood collection device 10 to the patient's specific finger size.

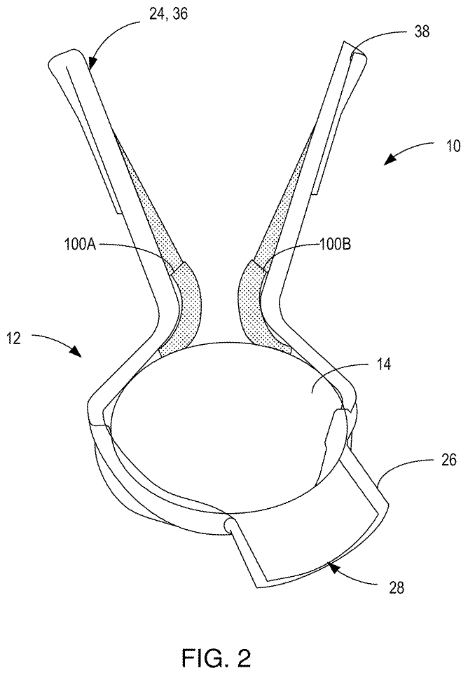

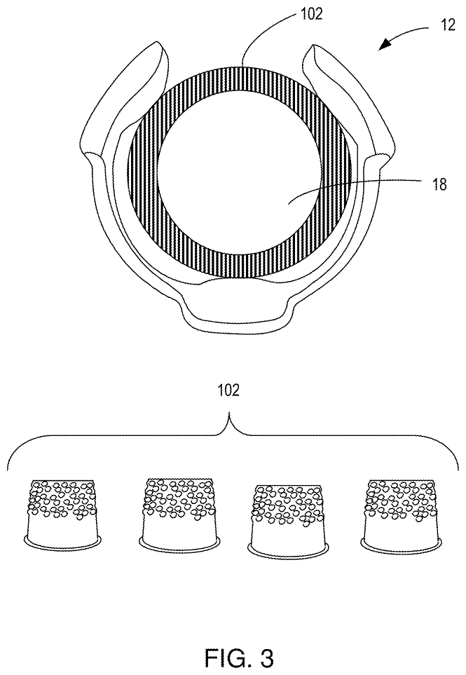

With reference to FIGS. 5 and 6, according to one non-limiting embodiment or aspect of the present disclosure, the blood collection device 10 may include a squeeze limiting element 110 to limit the amount or distance the wings 38 of the blood collection device 10 can be squeezed to reduce any pain a patient would experience when the wings 38 were over-squeezed. The squeeze limiting element 110 may be attached to any inner surface of the wings 38 so that as the wings 38 are squeezed, the opposing elements of the squeeze limiting element 110 will abut one another if the wings 38 are over-squeezed, thereby preventing any further closing of the wings 38 against the patient's finger 19. As shown in FIG. 6, the squeeze limiting element 110 can also be rotated downward to create added pressure against the patient's finger 19 held in the holder 12 to reduce any pain created by the wings 38 against the dorsal portion of the patient's finger 19. The squeeze limiting element 110 may be attached to the wings 38 via a living hinge. The blood collection device may also include a rear gripper 112 attached to the holder 12 via a living hinge 114. The living hinge 114 permits the rear gripper 112 to be rotated from a downturned position to an upturned position in which the rear gripper 112 covers and receives the holder 12.

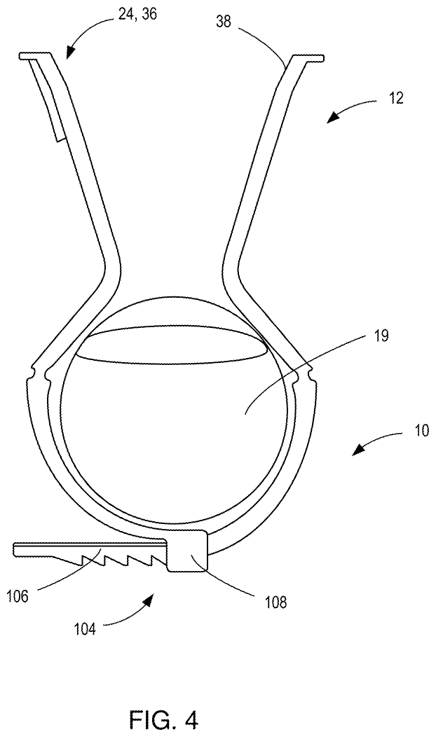

With reference to FIG. 7, according to one non-limiting embodiment or aspect of the present disclosure, the holder 12 may be designed with multiple, different sized finger holding grooves 116 that can accommodate numerous sizes for a patient's finger. In particular, a top portion of the holder 12 may include a finger holding groove 116 that is the largest of the grooves, while the bottom portion of the holder 12 may include a finger holding groove 116 that is the smallest of the finger holding grooves 116. When using the blood collection device 10, the patient may insert his/her finger into the holder 12 and will move his/her finger down along the finger holding grooves 116 until he/she finds a comfortable finger holding groove 116 to rest his/her finger. By ensuring a properly sized finger holding groove 116 is used for the patient, any pain experienced by the patient may be reduced or eliminated.

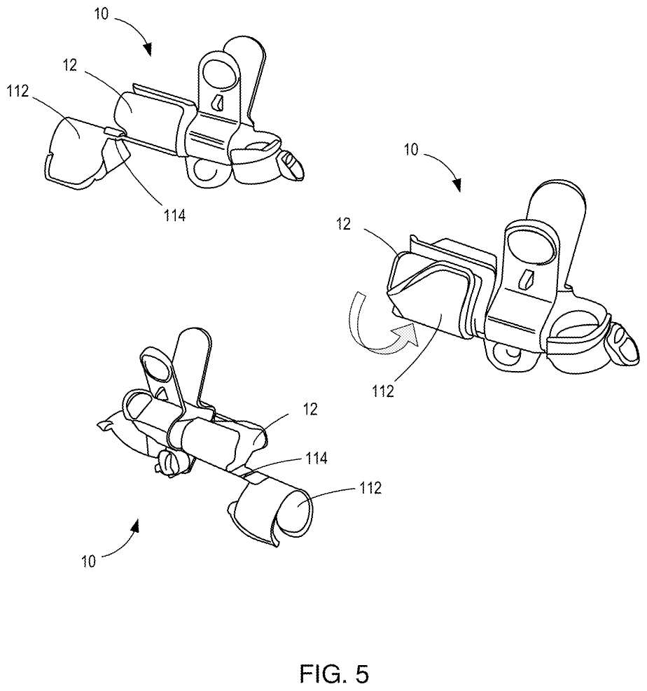

With reference to FIG. 8, according to one non-limiting embodiment or aspect of the present disclosure, the holder 12 may be designed with wings 118 designed and configured to be biased to a closed position. In this manner, the wings 118 are opened in a reverse order to the wings 38 discussed above that are squeezed into the closed position. The wings 118 shown in FIG. 8 are configured to rest in a closed position and can be squeezed to open the wings 118 to receive a patient's finger. Once the wings 118 are released, the wings 118 will close to rest against the patient's finger, thereby permitting a number of different sized patient's fingers to be used with the blood collection device 10. To open the wings 118, a user would grip the outer surfaces of the wings 118 and squeeze the wings 118 towards one another, thereby enlarging the channel defined by the wings 118 to receive the patient's finger.

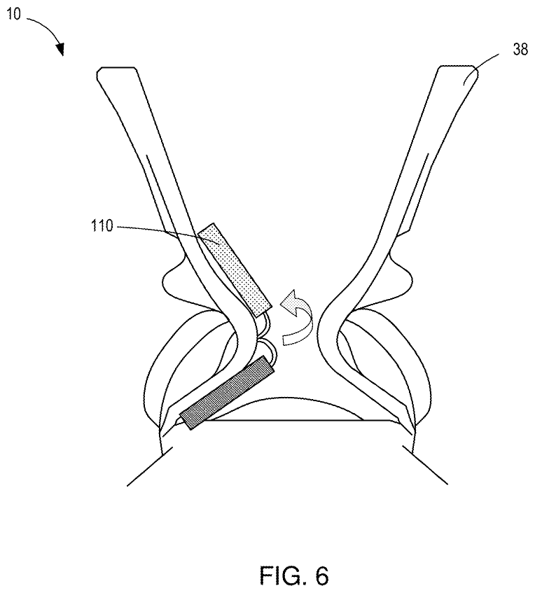

With reference to FIG. 9, according to one non-limiting embodiment or aspect of the present disclosure, the holder 12 may be designed with wings 120 that include cut-outs 122 to allow for fitting to different sizes of patient's fingers. In this configuration, inner wings 124 may be used to accommodate smaller sized patient fingers, thereby ensuring the wings 120 and holder 12 comfortable rest against the patient's finger when using the blood collection device 10. As also shown in FIG. 9, hinges 126 for the inner wings 124 may be adjusted based on the size of the patient's finger.

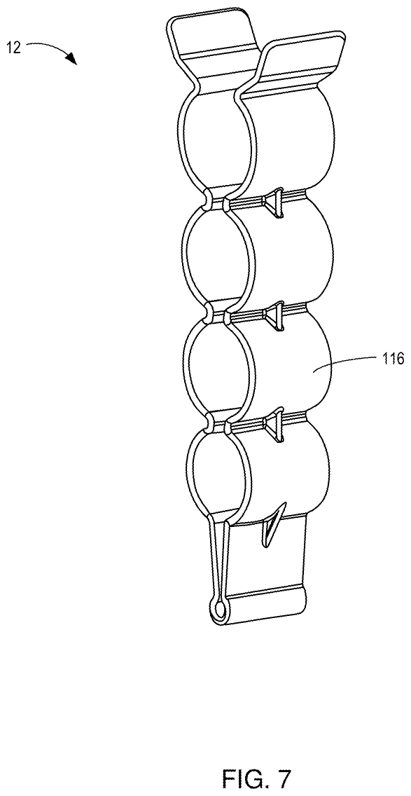

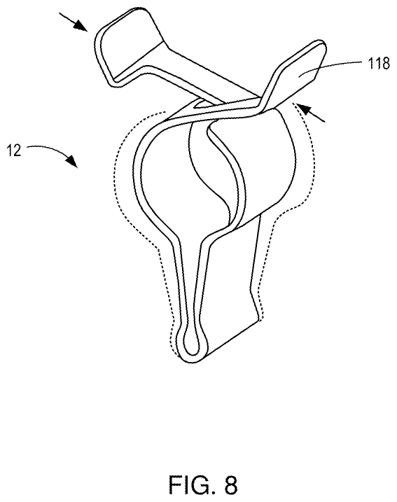

With reference to FIG. 10, according to one non-limiting embodiment or aspect of the present disclosure, the holder 12 may include an inflatable pad 128 positioned on a bottom surface of the holder 12. The inflatable pad 128 may be inflated or deflated to reduce the inner diameter of the channel defined by the holder 12, thereby permitting different sized patient fingers to be received in the holder 12. The inflatable pad 128 may be a bladder-type pad that can receive and dispel a fluid or liquid to increase or decrease the size of the inflatable pad 128. In one embodiment, the inflatable pad 128 may be filled with a heating gel to act as a heat source during blood collection, which promotes blood flow in the patient's finger. With reference to FIG. 11, according to one non-limiting embodiment or aspect, the holder 12 may be designed as a two part configuration including a first part that includes the inflatable pad 128 and wings 38 and a second part that includes the finger cuff and an adjustable band 130 to hold the patient's finger in the holder 12.

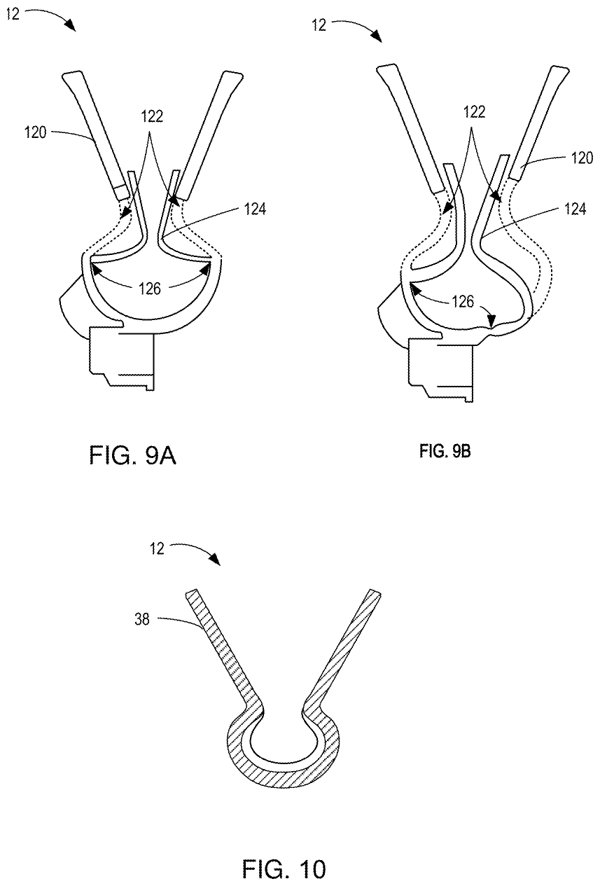

With reference to FIG. 12, according to one non-limiting embodiment or aspect of the present disclosure, the holder 12 may include a foam port 132 configured to permit an expanding foam 134 to be directed into the holder 12 to surround the patient's finger 19 to ensure the patient's finger comfortably rests in the holder 12. Based on the size of the patient's finger 19, the expanding foam 134 can be used so that a smaller patient finger 19 can be received and held in the holder 12 without moving within extra space of the holder 12. In one embodiment, the expanding foam 134 can be filled with a heating gel to act as a heat source to promote blood flow in the patient's finger 19.

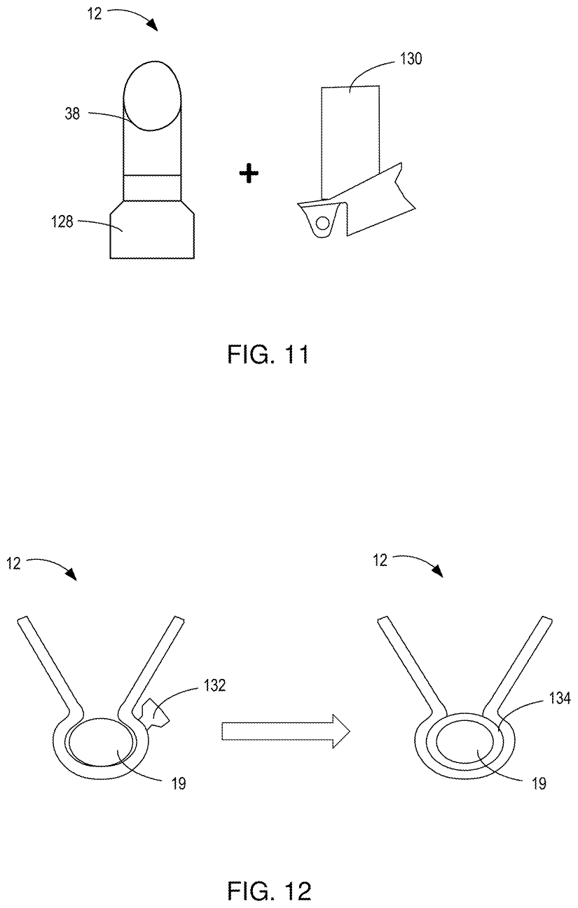

With reference to FIG. 13, according to one non-limiting embodiment or aspect of the present disclosure, the holder 12 may include a hook and look strap 136 to adjust the size of the holder 12 to accommodate a variety of patient finger sizes. The hook and loop strap 136 may be integrated into the holder 12 or, alternatively, may be separate hook and loop strap bands that are tightened on an exterior surface of the holder 12 to secure the holder 12 to the patient's finger.

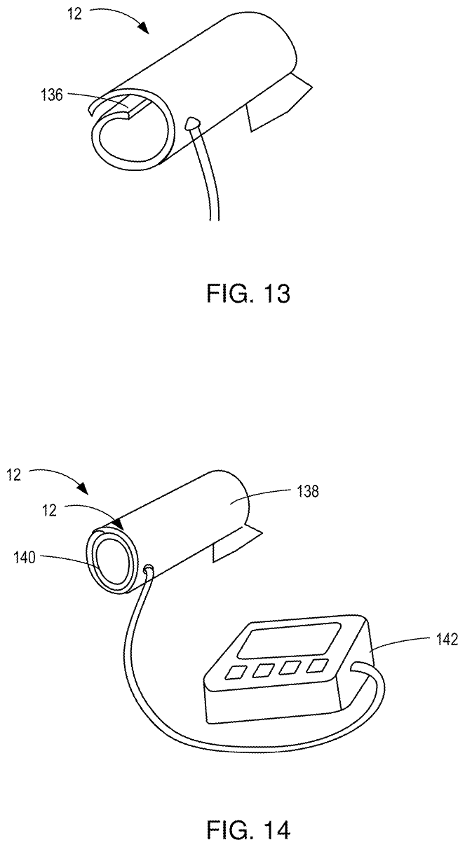

With reference to FIG. 14, according to one non-limiting embodiment or aspect of the present disclosure, the holder 12 may include a rigid outer shell 138 with an inflatable pad 140 provided in an inner channel of the rigid outer shell 138. The inflatable pad 140 may be filled with any fluid or liquid, such as air, to increase or decrease the diameter of the channel defined by the rigid outer shell 138. The inflatable pad 140 may be inflated and deflated to accommodate a number of different sizes of patient fingers and may be controlled by a control unit 142. The liquid or fluid used in the inflatable pad 140 may be heated to act as a heat source to promote blood flow in the patient's finger.

While different examples of the blood collection devices and methods are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A device for obtaining a blood sample, the device comprising:
a holder for receiving a sample source, the holder having an actuation portion and a port, wherein the actuation portion comprises a pair of opposing wings that are configured to be pumped to draw the blood from the sample source;
a container engagement portion connected to the holder; and
a pain reduction member provided on an inner surface of the holder.

2. The device of claim 1, wherein the pain reduction member comprises at least one elastomeric material positioned on the inner surface of the holder.

3. The device of claim 1, wherein the pain reduction member is positioned on an inner surface of the actuation portion of the holder.

4. The device of claim 2, wherein the at least one elastomeric material is positioned to contact a dorsal portion of the sample source held in the holder.

5. The device of claim 1, wherein the pain reduction member comprises a ring configured to cover an outer circumferential surface of the sample source.

6. The device of claim 5, wherein the ring is made of an elastomeric material.

7. The device of claim 5, wherein the ring comprises a plurality of ribs or bumps on an exterior surface thereof to grip the holder.

8. The device of claim 1, wherein the sample source is a finger.

9. The device of claim 8, wherein, with the finger received within the holder, the port is in communication with a portion of the finger.

10. (canceled)

11. A device for obtaining a blood sample, the device comprising:
a holder for receiving a sample source, the holder having an actuation portion and a port wherein the actuation portion comprises a pair of opposing wings that are configured to be pumped to draw the blood sample from the sample source;
a container engagement portion connected to the holder; and
a tightening arrangement provided on the holder to adjust a diameter of the holder that receives the sample source.

12. The device of claim 11, wherein the tightening arrangement includes a zip tie to decrease the diameter of the holder to tighten the holder to the sample source.

13. The device of claim 12, wherein the zip tie includes a zip tie strap on a first portion of the holder and a zip tie opening provided on a second portion of the holder

14. The device of claim 11, wherein the tightening arrangement is provided on a bottom surface of the holder.

15. The device of claim 11, wherein the sample source is a finger.

16. The device of claim 15, wherein, with the finger received within the holder, the port is in communication with a portion of the finger.

17. (canceled)

18. The device of claim 11, wherein the actuation portion includes a pumping member for applying pressure to the sample source.

19. A kit used for obtaining a blood sample, the kit comprising:
a blood collection device comprising:
a holder for receiving a sample source, the holder having an actuation portion and a port wherein the actuation portion comprises a pair of opposing wings that are configured to be pumped to draw the blood sample from the sample source; and
a container engagement portion connected to the holder; and
a pain reduction member provided separate from the blood collection device, the pain reduction member configured to minimize or eliminate pain experienced by the sample source held in the holder.

20. The kit of claim 19, wherein the pain reduction member comprises an elastomeric ring configured to surround an outer circumferential surface of the sample source.
